(19) United States
(12) Patent Application Publication
(10) Pub. No.: US 2006/0173426 A1
(43) Pub. Date: Aug. 3, 2006

(54) Aspiration system for orthopedic medical devices

(76) Inventors: Alex Urich, Rancho Santa Margarita, CA (US); Armand Maaskamp, Coto de Caza, CA (US)

Correspondence Address: IRELL & MANELLA LLP, 840 NEWPORT CENTER DRIVE, SUITE 400, NEWPORT BEACH, CA, 92660, US

(21) Appl. No.: 11/326,305

(22) Filed: Jan. 4, 2006

Related U.S. Application Data
(60) Provisional application No. 60/641,471, filed on Jan. 4, 2005.

(51) Int. Cl. A61M 1/00

(52) U.S. Cl. 604/317

(57) ABSTRACT

An aspiration system to be used in surgical procedures that require large amounts of sterile fluids circulated through the human body as for example orthopedic surgery. The aspiration system includes a filter assembly and a restrictive aspiration tube. The restrictive aspiration tube can be coupled to a vacuum source. The filter assembly filters out particles aspirated from a surgical site. The restrictive aspiration tube has an inner diameter between 0.05 and 0.3 inches and a length of at least 3 feet. These dimensions limit the flow that can be pulled through the human body, while allowing for adequate vacuum to aspirate from the surgical site.

### CROSS-REFERENCE TO RELATED APPLICATION

The present application claims priority to Provisional Application No. 60/641,471, filed on Jan. 4, 2005.

### BACKGROUND OF THE INVENTION

1. Field of the Invention

The present application relates to an aspiration system to be used in surgery where high amounts of sterile fluids are circulated through the human body as for example orthopedic surgery.

2. Prior Art

Some orthopedic medical procedures produce particles or other debris that must be removed from the body. To remove such particles the surgeon may couple an aspiration tube to the surgical site. The aspiration tube, which pulls the debris from the body, is typically connected to a canister, which is connected to a suction tube connected to wall suction. An irrigation fluid is introduced to the body to continuously irrigate the surgical site. To insure that the surgical site is properly distended during surgery the amount of irrigation fluid must be higher than the amount of aspirated fluid at any given time. An infusion pump is typically required to offset the high flow created by the hospital vacuum line. Infusion pumps are relatively expensive and are not always available to the surgeon. Additionally, vacuum surges are created when the suction line is obstructed and irrigation fluid cannot flow quickly enough to offset the outflow created by the hospital vacuum line.

In addition, the hospital suction line produces a flow rate in excess of 2 liter/minute which leads to a high consumption of sterile fluid during the procedure.

It would be desirable to provide an aspiration system that would eliminate the need for an infusion pump. It would also be desirable to provide an aspiration system that would limit vacuum surges in the system and reduce the circulation of fluid through the human body.

There have been developed flow restrictors that are used in ophthalmic procedures. For example, U.S. Pat. No. 6,478,781 issued to Urich et al. discloses a coiled tube that can be used to minimize pressure surges in an ophthalmic aspiration system. The tube has a length of at least 8 feet and a number of coils that create a fluidic resistance which minimizes vacuum surges. The recited inner diameter of the tube ranges from 0.06 to 0.1 inches, which is industry standard. Although effective, the coiled approach can only account for a limited pressure drop. Additionally, the coil does not contain a filter and thus is susceptible to occlusions within the coiled tube.

U.S. Pat. No. 6,599,271 issued to Easley and assigned to Syntec, Inc. discloses an ophthalmic aspiration system that has a flow restrictor and an in-line filter. Likewise, STAAR Surgical of Monrovia, Calif. sells an in-line ophthalmic filter under the name CRUISE CONTROL that contains a flow restrictor. The flow restrictors limit the vacuum surges within the ophthalmic aspiration system. These filter systems are not acceptable for use in orthopedic procedures.

### BRIEF SUMMARY OF THE INVENTION

An orthopedic aspiration system that includes a filter assembly and a restrictive aspiration tube. The restrictive aspiration tube has an inner diameter between 0.05 and 0.30 inches.

### BRIEF DESCRIPTION OF THE DRAWINGS

FIG. 1 is an illustration of an orthopedic medical system with an aspiration system;

FIG. 2 is a side view of an in-line filter of the aspiration system;

FIG. 3 is a cross-sectional view of the filter shown in FIG. 2;

FIG. 4 is a side view of an alternate embodiment of the in-line filter; and,

FIG. 5 is a cross-sectional view of the filter shown in FIG. 4.

### DETAILED DESCRIPTION

Disclosed is an orthopedic aspiration system. The orthopedic aspiration system includes a filter assembly and a restrictive aspiration tube. The restrictive aspiration tube can be coupled to a vacuum source. The filter assembly filters out particles aspirated from a surgical site. The restrictive aspiration tube has an inner diameter between 0.05 and 0.3 inches and a length of at least 3 feet. These dimensions limit the flow rate that can be pulled through the aspiration system, while allowing for adequate vacuum necessary to aspirate from the surgical site.

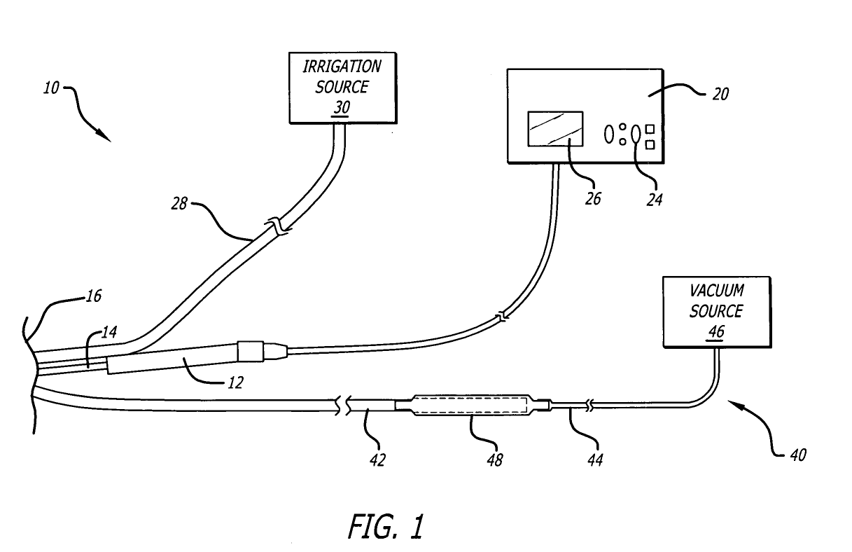

Referring to the drawings more particularly by reference numbers, FIG. 1 shows an embodiment of a medical system 10. The system 10 may include a hand piece 12 which has a cannula tip 14 that can be inserted into a body 16. The hand piece 12 is typically held by a surgeon who performs an orthopedic surgical procedure with the system. The cannula tip 14 may include some type of cutting element that cuts bone and/or tissue. This cutting action will create particles and other debris.

The handpiece 12 may be connected to a console 20 of the system 10. The console 20 may provide driving signals to the handpiece 12. The console 20 may have input knobs or buttons 24 that allow the surgeon to vary different parameters of the system 10. The console 20 may also have a readout display 26 that provides an indication of the power level, etc. of the system 10.

The system 10 may include an irrigation tube 28 that can be coupled to the surgical site. The irrigation tube is connected to an irrigation source 30. The irrigation source 30 may be a gravity fed bottle that contains an irrigation fluid that flows into the body 16 through the irrigation tube 28. The irrigation source 30 may include a pump to provide a relatively high flow of irrigation fluid to the surgical site. The medical system 10 may further have an aspiration system 40 that aspirates the irrigation fluid and debris out of the body 16. The aspiration system 40 may include an upstream aspiration tube 42 that is coupled to the body 16 and a restrictive aspiration tube 44 that is connected to a vacuum source 46. A filter assembly 48 is connected to the aspiration tubes 42 and 44. By way of example, the vacuum source 46 may be a vacuum line of a hospital. Alternatively, the vacuum source may be a vacuum pump. The vacuum source 46 creates a negative pressure within the aspiration system 40 to induce a flow of irrigation fluid and debris out of the body 16. The vacuum source 46 is configured so that the flow rate through the irrigation tube 28 is slightly greater than the flow rate through the aspiration system 40.

The restrictive aspiration tube 44 has a relatively large fluidic resistance to create a large pressure drop and inertia in the aspiration system 40. The pressure drop reduces the free unobstructed flow and the large inertia minimizes instantaneous changes in the flow rate of the irrigation fluid flowing through the aspiration tube 44. Thus if the aspiration system 40 is opened the large fluidic resistance of the tube 44 will restrict the variation in the aspiration line and minimize vacuum surges.

The second aspiration tube 44 has an inner diameter between 0.05 and 0.30 inches and a length of at least 3 feet. It is desirable to create a fluidic resistance that causes a pressure drop approximately equal to the maximum pressure of the vacuum source. This will minimize the change in flow rate within the aspiration system in the event a maximum pressure occurs because of an occlusion.

The fluidic resistance of the restrictive aspiration tube 44 limits the vacuum pressure within the aspiration system 40. This vacuum limit may allow the irrigation source 30 to be a gravitation bag that does not require an infusion pump as found in the prior art. Eliminating the infusion pump reduces the complexity and cost of the system.

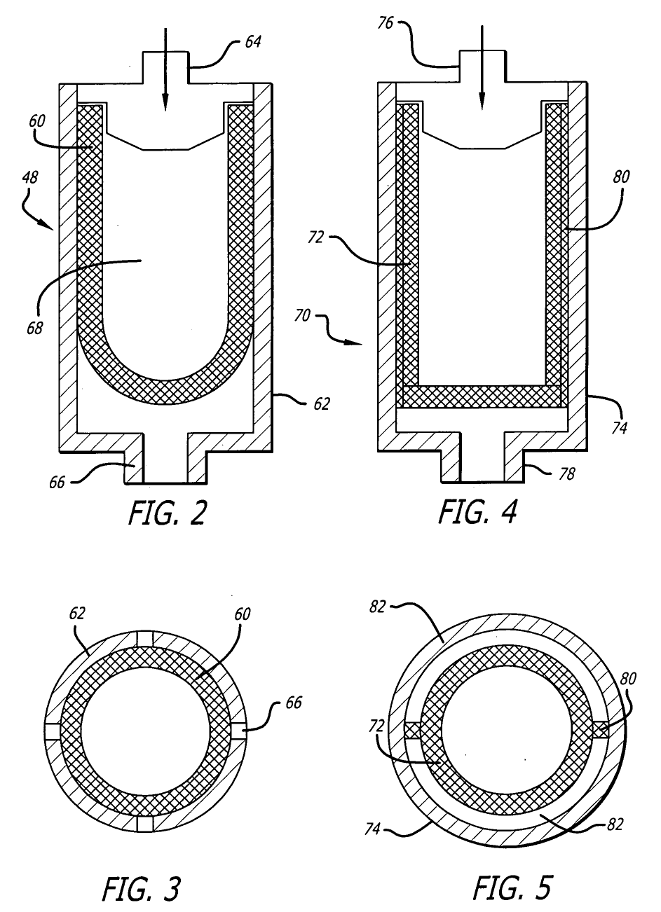

FIGS. 2 and 3 show an embodiment of an in-line filter assembly 48. The in-line filter 48 may include a filter mesh 60 located within a filter housing 62. The filter housing 62 may be roughened to reduce the adhesion of air bubbles to the inner wall of the housing. By way of example the inner wall of the housing 62 may have a roughness between 5 to 500 microns. The filter assembly 48 may have a fluid volume ranging from 1 to 25 cc. The housing 62 may include integral luers 64 and 66 that are connected to the first 42 and second 44 aspiration tubes (not shown), respectively. The filter mesh 60 may initially be a flat sheet that is bent and pushed into the filter housing 62 to create a U-shape filter. The filter 60 may have a mesh opening area no greater than 0.01 per square inch.

The filter housing 62 may have longitudinal grooves 66 as shown in FIG. 3 that allow fluid to flow through the filter assembly when particles fill the inner chamber 68 of the filter mesh. Without such grooves particles captured by the filter mesh 62 may occlude the mesh and limit the life of the filter during a procedure.

FIGS. 4 and 5 show an alternate embodiment of the filter assembly 70. The assembly includes a filter mesh 72 inside a filter housing 74. The filter 72 may have a mesh opening area no greater than 0.01 per square inch. The housing 74 may have luers 76 and 78 connected to the tubes 42 and 44 (not shown), respectively. The housing 74 may be roughened and have a fluid volume the same or similar to the filter described and shown in FIGS. 2 and 3.

The filter mesh 72 may include a pair of ears 80 that create channels 82 between the mesh 72 and the filter housing 74. The channels 82 allow for fluid to flow even when particles are being captured by the filter mesh 72.

The orthopedic aspiration system 40 can filter particles and minimize vacuum surges without introducing complicated parts or increased cost to the system.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. An orthopedic aspiration filter system, comprising:
a restrictive aspiration tube having an inner diameter between 0.05 and 0.30 inches; and,
a filter assembly coupled to said restrictive aspiration tube.

2. The system of claim 1, wherein said restrictive aspiration tube has a length of at least 3 feet.

3. The system of claim 1, further comprising an upstream aspiration tube coupled to said filter assembly.

4. The system of claim 1, wherein said filter assembly includes a filter located within a filter housing.

5. The system of claim 4, wherein said filter has a mesh opening area no greater than 0.01 per square inch.

6. An orthopedic aspiration filter system, comprising:
a filter assembly; and
restriction means for limiting a vacuum pressure within said filter assembly and the system.

7. The system of claim 6, wherein said restriction means includes a restrictive aspiration tube having an inner diameter between 0.05 and 0.30 inches.

8. The system of claim 7, wherein said restriction aspiration tube has a length of at least 3 feet.

9. The system of claim 6, further comprising an upstream aspiration tube coupled to said filter assembly.

10. The system of claim 6, wherein said filter assembly includes a filter located within a filter housing.

11. The system of claim 10, wherein said filter has a mesh opening area no greater than 0.01 per square inch.

12. A method for operating an orthopedic aspiration system, comprising:
aspirating a fluid and particles through a first tube, a filter assembly and a restrictive aspiration tube, the restrictive aspiration tube having an inner diameter between 0.05 and 0.30 inches.

13. The method of claim 12, wherein the filter assembly filters the particles.

14. The method of claim 12, wherein the restrictive aspiration tube has a length of at least 3 feet.

15. The method of claim 12, wherein the particles are created by a hand piece.
